(19) United States
(12) Patent Application Publication
(10) Pub. No.: US 2026/0112480 A1
(43) Pub. Date: Apr. 23, 2026

(54) SYSTEM AND METHOD FOR ASSESSING, TRACKING AND MODIFYING HEALTH AND PERSONALIZING IT BASED ON MUSIC, MEDIA CONTENT AND DIVERSITY METRICS

(71) Applicant: CHIME HEALTH AI INC., Newark, DE (US)

(72) Inventors: David M. GREENBERG; Igor RADOVANOVIC, Brod (BA)

(73) Assignee: CHIME HEALTH AI INC., (US)

(21) Appl. No.: 19/117,046

(22) PCT Filed: Sep. 10, 2023
(86) PCT No.: PCT/IB2023/058957

§ 371 Date: Mar. 30, 2025

Related U.S. Application Data
(60) Provisional application No. 63/411,173, filed on Sep. 29, 2022.
(60) Provisional application No. 63/438,256, filed on Jan. 11, 2023.

(51) Int. Cl. G16H 40/63

(52) U.S. Cl. CPC G16H 40/63

(57) ABSTRACT

Disclosed herein is a method for assessing the health of a user, the method performed by at least one processor configured to execute one or more programs stored in a memory, the one or more programs comprising instructions to obtain input data associated with the user, extract at least one characteristic associated with said input data, determine a health score based on variation of predetermined values, determine whether said health score satisfies a threshold associated with at least one health condition, generate a personalized message identifying at least one health condition, and provide said personalized message and said health score to an output device.

### RELATED APPLICATIONS

The present application claims priority from U.S. provisional patent application Ser. No. 63/411,173 filed on Sep. 29, 2022, and U.S. provisional patent application Ser. No. 63/438,256 filed on Jan. 11, 2023.

### FIELD OF THE INVENTION

The present disclosure generally relates to personalizing the health and user experience on media streaming, and in particular, to health assessment, health tracking and personalized health modification that is assigned to the user based on music and media preferences, music and media content consumption, and diversity metrics.

### BACKGROUND

Access to electronic media, such as music and video content, has expanded dramatically over time. As a departure from physical media, content providers have adapted and adopted to providing most content by streaming media to electronic devices. Such streaming services improve the convenience with which users can consume and experience such content. In addition, the development of personalized healthcare (and personalized medicine) has also expanded drastically over time. As a departure from in-person healthcare, digital health and telehealth platforms have created electronic possibilities for assessing, tracking, and modifying health and wellness.

### SUMMARY

Accordingly, there is a need for systems and methods for assessing, tracking, and modifying health, and personalizing it, based in accordance with music and media preferences, music and media content consumption, and diversity metrics of the user. Music and media preferences, for example, may be a good indication of a user's health status. Determining a user's health status (across multiple domains including mental health, neurological health, social health, and physical health) from their preferences and consumption, gives media-providing services and digital health services a sophisticated tool for assessing and tracking user health, and more generally, user experience.

In addition, based on user health information, media content (including music) can be personalized and recommended to users to modify (e.g., improve) their health and wellness and achieve their goals. Media content (including music) can also be generated in a personalized way (e.g., via artificial intelligence) for a user in such a way that will modify their health. Modifying their health using media content provides media-providing services (e.g., streaming services) and digital health services a sophisticated tool for modifying user health.

The following embodiments and aspects thereof are described and illustrated in conjunction with systems, tools and methods which are meant to be exemplary and illustrative, not limiting in scope.

There is provided, in accordance with an embodiment, a method for assessing the health of a user, the method performed by one or more processors configured to execute one or more programs stored in a memory, the one or more programs including instructions to obtain input data associated with the user, extract one or more characteristics associated with the input data, determine a health score based on variation of predetermined values, determine whether the health score satisfies a threshold associated with one or more health conditions, generate a personalized message identifying one or more health conditions, and provide the personalized message and the health score to an output device.

In some embodiments, the input data includes a user musical preference questionnaire input, a playlist history, self-reported measure of musical preferences, user recorded music, demographic information, a user written text about music, entertainment history and preference, media history and preference, music selected for social media posts, music listened to through social media, and a combination thereof.

In some embodiments, the input mental and physical health assessments and personality questionnaires.

In some embodiments, the threshold is associated with a mental health condition.

In some embodiments, the threshold is associated with a neurological condition.

In some embodiments, the threshold is associated with a social condition.

In some embodiments, the threshold is associated with a physical condition.

In some embodiments, the one or more characteristics that describes the input data include values associated with categories selected from the group consisting of genre, artist, popularity, playlist co-occurrence and emotion.

In some embodiments, personalized message presents a spectrum of a health condition and presenting where on the spectrum the user is categorized.

In some embodiments, the health condition is a combination of characteristics associated with one or more health conditions.

There is further provided, in accordance with an embodiment, a system configured to assess a health condition of a user, the system including a user interface having one or more input devices and one or more output devices, a client device having one or more processors, a memory storing one or more programs for execution by the one or more processors, the one or more programs including instructions to, obtain input data associated with the user, extract one or more characteristics associated with the input data, determine a health score based on variation of characteristics extracted, determine whether the health score satisfies a threshold associated with one or more health conditions, generate a personalized message identifying one or more health conditions, and provide the personalized message to an output device;

In some embodiments, the input data includes a user musical preference questionnaire input, a playlist history, self-reported audio-based measures of musical preferences, user recorded music, demographic information, a user written text about music, entertainment history and preference, media history and preference, music selected for social media posts, music listened to through social media, and a combination thereof.

In some embodiments, the input includes mental and physical health assessments and personality questionnaires.

In some embodiments, the system further includes one or more media servers having a server memory configured to store the playlist history.

In some embodiments, the threshold is associated with a mental health condition.

In some embodiments, the threshold is associated with a neurological condition.

In some embodiments, the threshold is associated with a social condition.

In some embodiments, the threshold is associated with a physical condition.

In some embodiments, the one or more characteristics that describes the input data include values associated with categories selected from the group consisting of genre, artist, popularity, playlist co-occurrence and emotion.

In some embodiments, the personalized message presents a spectrum of a health condition and presenting where on the spectrum the user is categorized.

In some embodiments, the health condition is a combination of characteristics associated with one or more health conditions.

There is further provided, in accordance with an embodiment, a system configured to provide a media content recommendation to improve a health of a user, the system including a user interface having at least one input device and at least one output device, a client device having at least one processor, a memory storing one or more programs for execution by the at least one processor, the one or more programs including instructions to obtain a first set of input data facilitating generating a prediction of media content recommendation, analyze the first set of input data to determine the media content that may improve the health condition and status of the user, generate a media content recommendation, record modification data during and after the user has consumed the recommended media content, generate a personalized message indicating the health condition and status of the user after consumption of the recommended media content, and providing the personalized message to the at least one output device.

In some embodiments, the processor is further configured to generate a new media content recommendation according to the modification data.

In some embodiments, the processor is further configured to personalized media content according to the first set of input data.

In some embodiments, the first set of input data includes the input data and the health score.

In some embodiments, the personalized message provides a medication recommendation to correspond to the media content recommendation.

In some embodiments, the recommendation can includes a treatment plan for clinical psychology, psychotherapy, and music therapist that is provided directly to a treatment provider.

In some embodiments, the processor is further configured to generate a recommendation model, update the recommendation model according to the improvement data, and store the recommendation model.

### BRIEF DESCRIPTION OF THE DRAWINGS

Some non-limiting exemplary embodiments or features of the disclosed subject matter are illustrated in the following drawings.

FIG. 1 is a schematic illustration of a system for assessing a health condition and status of a user and for delivery of media content, according to certain exemplary embodiments;

FIG. 2 is a schematic illustration of a client device of the system from FIG. 1, according to certain exemplary embodiments;

FIG. 3 is a schematic illustration of a media server of the system from FIG. 1, according to certain exemplary embodiments;

FIG. 4 is a block diagram outlining operations of a method for assessing a health condition and status of a user, according to certain exemplary embodiments; and

FIG. 5 is a block diagram outlining operations of a method for providing a media content for improving a health condition or status of a user, according to certain exemplary embodiments.

Identical, duplicate, equivalent or similar structures, elements, or parts that appear in one or more drawings are generally labeled with the same reference numeral, optionally with an additional letter or letters to distinguish between similar entities or variants of entities, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

References to previously presented elements are implied without necessarily further citing the drawing or description in which they appear.

### DETAILED DESCRIPTION

Disclosed herein is a system and method for determining a health condition and status of a user, according to certain exemplary embodiments.

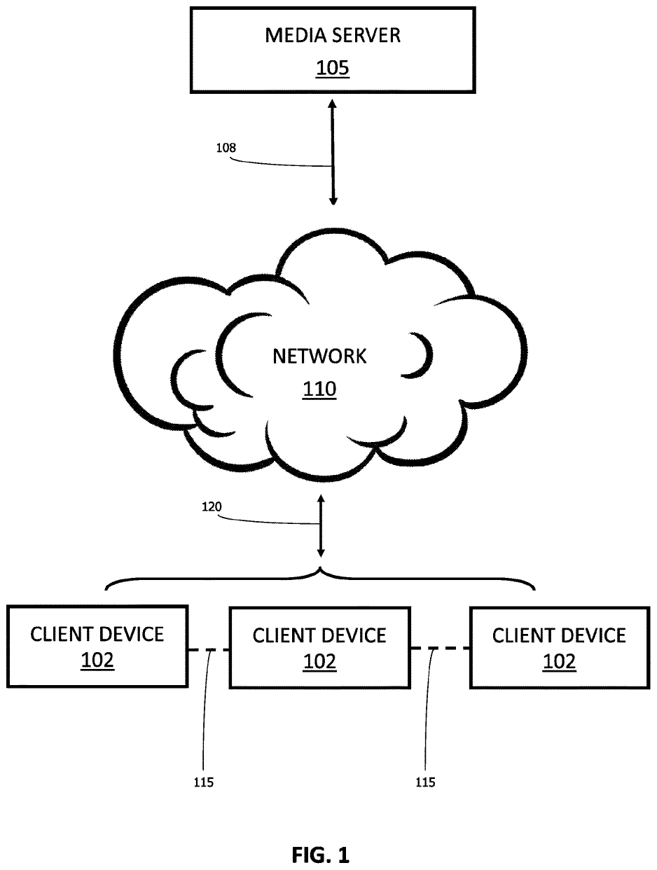

Referring now to FIG. 1, schematically illustrates a system 100 for assessing, detecting, screening and determining a health condition and status of a user, for delivery of media content, and for providing media content to the user to improve, change, moderate, reinforce, and track the health condition and status of the user, according to certain exemplary embodiments. System 100 includes one or more client devices 102, illustrated as three instances of client devices 102, representing any number of client devices 102, as indicated by dashed line 115. In some embodiments, client devices 102 can include smartphones, tablets, televisions, audio systems, desktops, or the like. Client devices 102 are connected or linked to a network 110 by any communication facility or facilities included in system 100 as schematically illustrated by arrow 120, which facilitate the data flow from one or more client devices 102 to network 110. In some embodiments, client devices 102 can include smartphones, tablets, televisions, audio systems, desktops, or the like. System includes one or more media servers 105, which are connected to network 110 by any communication facility or facilities included in system 100 as schematically illustrated by arrow 108, which facilitate the data flow from media servers 105 to network 110. The media server 105 are associated with a media providing service.

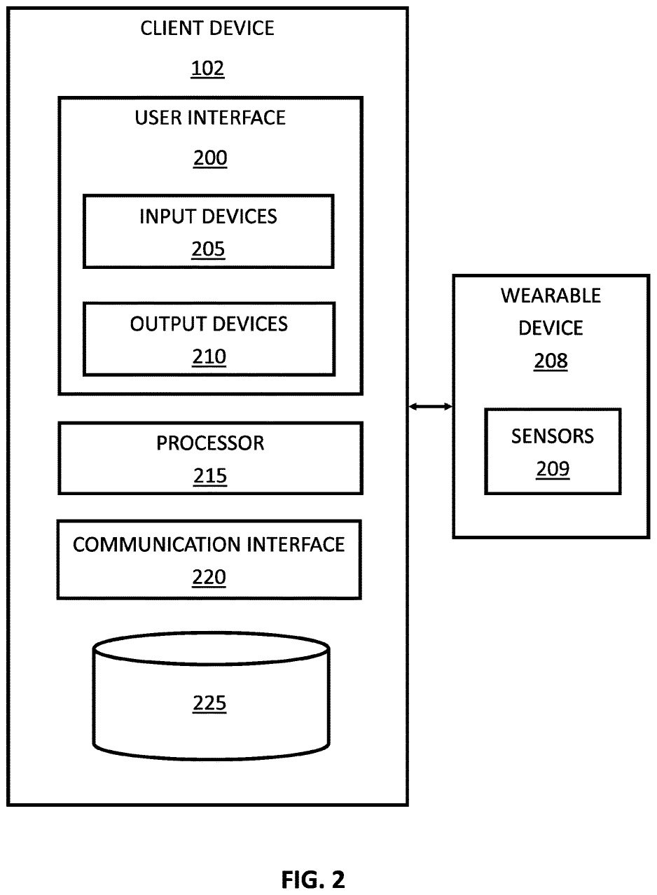

Referring to FIG. 2, schematically illustrates client device 102, according to certain exemplary embodiments. Client device 102 is configured to execute a computer program product, the computer program product includes a non-transitory computer-readable storage medium having program code embodied therewith, the program code executable by one or more hardware processors 215. Client device 102 includes a user interface 200 configured to facilitate interaction between client device 102 and a user.

User interface 200 includes input devices 205 configured to record inputs provided by a user. For example, input devices 205 can include a keyboard, a mouse, a microphone, or the like. In some embodiments, input device 205 can be associated with one or more wearable devices 208, which include one or more sensors 209 configured to record biometric data of the user. Wearable device 208 transmits the biometric data to client device 200 to enable processor 215 to monitor the change in the health condition and status of the user while the user is consuming the media content. User interface 200 includes output devices 200 configured to provide an output to a user. For example, out devices can include one or more speakers that output audio content, a display for displaying image and video content, a health condition assessment, or the like.

Client device 102 includes a communication interface 220 configured to facilitate communication with network 110 (FIG. 1) and with one or more media servers 105 (FIG. 1), thereby providing the requested media content to a user of client device 102. Client device 102 includes a memory 225, which includes high-speed random-access memory, such as DRAM, SRAM, DDR RAM, or other random-access solid-state memory devices; and may include non-volatile memory, such as one or more magnetic disk storage devices, optical disk storage devices, flash memory devices, or other non-volatile solid-state storage devices, or the like. In some embodiments, memory 225 can include remote storage devices. In some embodiments, memory 225 stores the follow programs, modules, data structures or a subsets or superset thereof: an operating system, a network communication module, a user interface module, a media application, and other applications.

The operating system includes procedures for handling various basic system services and for performing hardware-dependent tasks. One or more network communication modules for connecting client device 102 to other computing devices via one or more networks interfaces connected to one or more networks 110. The user interface module receives commands and/or inputs from a user via the user interface 200 and provides outputs for playback and/or display on user interface 200. The media application associated with and for accessing a media-providing service of a media content provider such as media server 105, for example, including a media player, a streaming media application, or any other appropriate application or component of an application. The media application facilitates browsing, receiving, processing, presenting, and requesting playback of media, such as audio tracks or videos. In some embodiments, the media application can also facilitate monitoring, storing, and/or transmitting data associated with user interaction, for example to media server 105. The media application also includes the following modules, sets of instructions, or a subset or superset thereof such as a media content browsing module, a content items module and a web browser application.

The media content browsing module facilitates providing controls and/or user interfaces that enable a user to navigate, select for playback, and otherwise control or interact with media content, whether the media content is stored or played locally or remotely. The content items module is configured for storing media items for playback. The web browser application is configured to facilitate accessing, viewing, and interacting with web sites and other applications.

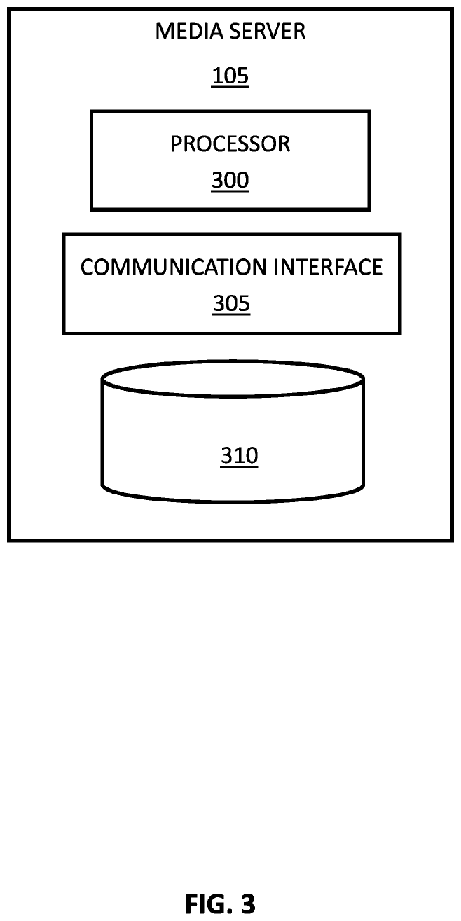

FIG. 3 schematically illustrates media server 105, according to certain exemplary embodiments. Media server 105 is configured to execute a computer program product, the computer program product includes a non-transitory computer-readable storage medium having program code embodied therewith, the program code executable by one or more hardware processors 300. includes a communication interface 305 configured to facilitate communication with network 110 (FIG. 1) and with client devices 102 (FIG. 1).

Media server 105 includes a memory 310 which includes high-speed random-access memory, such as DRAM, SRAM, DDR RAM, or other random-access solid-state memory devices; and may include non-volatile memory, such as one or more magnetic disk storage devices, optical disk storage devices, flash memory devices, blockchain or other non-volatile solid-state storage devices, or the like. In some embodiments, memory 310 can include remote storage devices. In some embodiments, memory 310 stores the follow programs, modules, data structures or a subsets or superset thereof: an operating system, a network communication module, a server application module, an authentication module, a media request module, a content personalization module, a server data module and other applications.

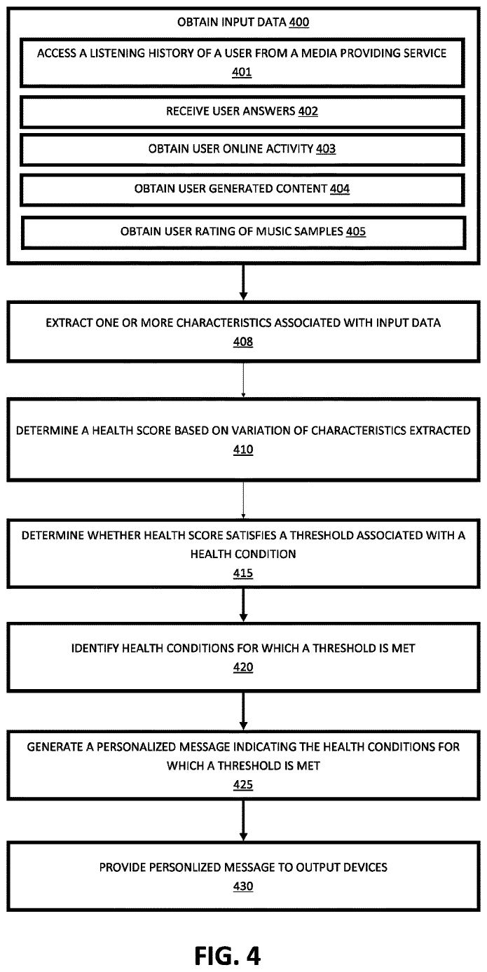

FIG. 4 is a block diagram outlining operations of a method for generating an assessment of a health condition and status of a user, according to certain embodiments.

In operation 400, processor 200 (FIG. 2) and/or processor 300 (FIG. 3) obtains input data. The input data can be accessed from multiple sources. Processor 300 is configured to access a listening history of the tracks consumed by the user (operation 401). The listening history can include multiple listening sessions in which each listening session includes one or more tracks consumed by the user. In some embodiments, the listening history can indicate an order in which the tracks are consumed by the user and the number of times each track in the listening history is consumed by the user. In some embodiments, the listening history can include information about the tracks consumed, such as tempos, genres, artist names, or the like. In some embodiments, input data can include the input data includes a user musical preference questionnaire input, a playlist history, self-reported measure of musical preferences, user recorded music, demographic information, a user written text about music or the like. In some embodiments, user recorded music can include live performances, written or performed compositions, or the like. In some embodiments, the input data can include music used or selected for social media posts or listened to through social media. In some embodiments, user input data can include entertainment or media history or preferences, such as movies, films, television content, art, or the like.

Each track in the listening history is associated with characteristics that describe the track. For example, characteristics can include one or more categories, such as the artist name, genre, emotional reaction, popularity, playlist co-occurrence, emotional attributes (e.g., sad or happy, et.), sonic attributes (e.g., percussiveness, tempo, volume, etc.), or the like. In some embodiments, the characteristics can be associate, or represented as a vector combination, of one or more characteristics.

Processor 200 and/or processor 300 is configured to receive user answers (operation 402). The user is presented with a questionnaire via output devices 210 (FIG. 2), In some embodiments, the questionnaire can ask users to listen to one or more musical tracks and/or excerpts and then requests that the user indicate how much they like each track or excerpt on a rating scale or the like. In some embodiments, the responses to different questionnaires can be combined to provide more accurate health scores. The responses to the questions can include answers regarding mental health (e.g., Patient Health Questionnaire-9), physical health, or the like. In some embodiments, the questionnaires combined can be a musical preference questionnaire with a personality questionnaire such as a NEO Personality Inventory-3, Big Five Inventory, or Myers-Briggs Type Indicator questionnaires. The user can provide answers to the questionnaire via input devices 205 (FIG. 2).

Processor 200 and/or processor 300 is configured to obtain user activity (operation 403). In some embodiments, processor 200 and/or processor 300 can obtain a user online activity, for example, collecting a user history from social media, in which the user posted about musical preferences, links to media, reviews of media, or the like. In some embodiments, processor 300 can obtain data from a wearable device such as a Fitbit or the like. In some embodiments, processor 300 can obtain an electronic health record (“EHR”), electronic medical record (“EMR”) or the like of a user to facilitate associating a musical preference with a health condition by correlating the medical and health record of the user with the music the user prefers to listen to.

Processor 200 and/or processor 300 is configured to obtain user generated content (operation 404). In some embodiments, processor 200 and/or processor 300 obtains user generated content such as musical content composed by the user, or the like.

Processor 200 and/or processor 300 is configured to obtain a user rating of musical samples (operation 405). For example, processor 200 and/or processor 300 presents the user with a predetermined number of short musical samples within a predetermined time range. For example, 25-30 seconds audio samples presenting a variety of musical styles, genres, attributes, and the like. The user then provides a rating of each sample. The rating can be a preference rating, or the like, using a Likert scale (e.g., with answer choices ranging 0 (dislike extremely) to 10 (like extremely)).

In operation 408, processor 200 and/or processor 300 extracts one or more characteristics associated with the input data. The one or more characteristics can include values associated with categories selected from the group consisting of genre, artist, popularity, playlist co-occurrence, emotion, or the like. In some embodiments, the one or more characteristics are stored in the metadata of the input data. In some embodiments, processor 300 can be configured to extract the one or more characteristics from the input data within a predetermined time window.

The health condition and status of a user may relate to the user's habits with respect to consumption of media content and activities. A variety of mood, genre, demographic, physical, mental and emotional condition and behavioral variables can correspond to different personality traits of the user. Thus, it is possible to identify a health condition and status of the user based on the media content the user consumes and the context in which the user consumes the content.

For example, music that is slow and sorrowful tends to be preferred by users who may suffer from depression. Users who do not suffer from depression tend to prefer music that is uplifting and upbeat. In addition to the genres of the music, the health condition and status of the user may also be related to a user's preferences for broad media content types, features and emotional content. An accumulation of behavior of the user, such as listening or viewing history of media content (e.g., music and videos respectively) including content choices and patterns of interaction with the media-providing service, may be used to define the health condition and status as well as provide a recommendation to modify the health condition and status.

Before assigning the health condition and status of the user, a health condition and status model may be built to relate information about a user and the user's media content interaction patter to the health condition and status. The health condition and status model may be based on a variety of data sources related to users' demography (e.g., age, gender), media content taste, and interactions with the media-providing service (e.g., with an application on the client device 102). These data sources may be measured over a period of time (e.g., months). The model may also include the user's media preferences (e.g. musical taste, video preferences), which may be based on data from the users' media content history.

In some embodiments, collaborative filtering vectors may be used to represent subtle systematic dependencies within the input data. Their coverage may be both great and very granular. They may capture subtle differences and similarities between different inputs provided that are not easily described in terms of a specific category, for example, lyrics, sonic attributes, genre and the like. Collaborative filtering vectors can provide important semantic value. For example, the collaborative filtering vectors follow a word2vec model. A word2vec model may take textual input and produce a vector space, where each unique word of the input data may be assigned a corresponding vector in the vector space.

For example, if tracks in the listening history are characterized by genre, for any genre, style, attribute cluster or the like, that tracks closest in vector space to tracks known to be in that genre may also be tracks in that genre. Thus, for two tracks that have the same genre and similar attributes (e.g., tempo and emotional characteristics), the two vectors representing the tracks will be closely located in the latent space for the vectors. Where there is a plurality of characteristics (e.g., corresponding to categories such as genre, emotion, playlist co-occurrence, etc.) that describe a given track, the collaborative filtering vector for that track can be used to capture these characteristics.

Machine learning models, such as a Lasso regression model, a clustering model, a decomposition model, a classification model, or the like, using the same set of metrics may be used to predict the numerical values of each of the health conditions and status. In some embodiments, a plurality of machine learning models (e.g. classification, clustering, and decomposition) may be used to predict the health conditions and status. For each model, cross-validation (e.g., a 10-fold cross-validation) and/or conformal prediction may be used to test the out-of-sample accuracy of the model. This may indicate which of the health conditions and status are most predictable based on the data. Further, each health condition and status may be characterized by the presence of a specific variable.

In some embodiments, the health conditions and status may also have correlations with other metrics, such as discovery metrics and diversity metrics. In some embodiments, discovery metrics can include new media content consumed by the user, the frequency of new media content being consumed, or the like. Furthermore, in some embodiments, diversity metrics can include demographics, income, gender, age, or the like. For example, depression may correlate to maintaining a steady level of diversity across all time scales. The model may also be based on deep learning or trained on demographic segments. Other inputs, such as longer consumption windows or additional metrics, may also be added to the model.

In operation 410, processor 200 and/or processor 300 determines a health score based on variation of the one or more values. In some embodiments, the health score can include a music score that is based on one or more characteristics extracted. The music score can include metrics for determining which health conditions are associated with the user.

The health score may be an aggregate health score including the music score. An aggregate health score can be defined as the average distance (e.g., cosine similarity or sine similarity in vector space) between every pair of tracks, sequential and non-sequential, in a set of tracks (e.g., the set of all tracks) listened to by a user in a given time window (e.g., the listening history or a portion thereof). If the number of tracks in the time window is sufficiently large (e.g., satisfies a threshold), this number can be accurately approximated by uniformly sampling pairs of tracks from the whole set.

In some embodiments, the health score can be a sequential health score. The sequential health score is based on the ordered sequence of tracks that a user listened to and may be calculated by measuring the average distance between each pair of consecutive tracks. This metric may capture a sense of cohesion or “orderliness” of listening versus chaotic and inconsistent listening, where a cohesive listening pattern may correspond to a lower diversity than an inconsistent listening pattern. The sequential health score can indicate how cohesive the style of listening is for the user within one session or part of a session as compared with how different the user's sessions may be. Although this metric may be heavily affected by the usage of shuffle, even in those cases it can be expect a user to jump randomly among similar tracks from a given album or playlist, and then incur a bigger jump distance (e.g., as defined by the distance between the vectors corresponding to successive tracks) when switching to another playlist or album.

In some embodiments, the vector distance between attributes of the music is analyzed by the correlating characteristics. For example, the health score can also be determined by the distance of the preferred great and gradual (style and nuanced) musical attributes that are preferred or consumed by a user. Furthermore, the interaction of the music data with other data (e.g., on personality traits) increases the accuracy of the health score.

In some embodiments, the health score can include an amount of media consumed by the user, rating of music or other media in the life of the user, history of prior musical training, musical experience of the user, or the like.

In operation 415, processor 200 and/or processor 300 determines whether the health score satisfies a threshold associated with a health condition. Health conditions can include different types of health conditions, such as a mental condition, a neurological condition, a social condition, a physical condition, and the like. A health condition can include the diagnosis, such as, the current and/or prior experienced symptoms of that condition, or having once had that condition, but no longer experiencing symptoms, the severity of the condition, and the age of onset of the condition. Health conditions in each category, such as a mental health condition, can include all possible conditions in that category. For example, mental health condition can include disorders commonly classified as anxiety disorders, mood disorders, substance use disorders, neurological disorders, personality disorders, psychotic disorders, eating/feeding disorders, trauma-related disorders, and substance use disorders. Below is a table listing exemplary conditions across varied health categories:

TABLE 1
List of Health Conditions

| | | Physical health |
|---|---|---|
| Mental health and/or neurological | Mental health and/or | disease, condition, |
| conditions/disorders | social behavior | and/or status |
| ADHD | Alexithymia | Allergies |
| Anorexia Nervosa | Number Of Autistic | Arthritis |
| Autism | Traits | Cancer |
| Bipolar Disorder | Depression Severity | Cardiovascular |
| Borderline | Emotion Regulation | Disease |
| Personality | Empathy | Chronic Migraine |
| Disorder | Mindfulness | Headaches |
| Bulimia Nervosa | Mood State and Trait | Dementia |
| Depression | Posttraumatic Stress | Diabetes |
| Drug addiction | Childhood Trauma | High Cholesterol |
| Dyslexia | History (Including | Hypertension/High |
| Epilepsy | Violence, Sexual | Blood Pressure |
| General Anxiety Disorder | Abuse, Loss of | Kidney Disease |
| Narcissistic Personality Disorder | Loved One, Parental | Multiple Sclerosis |
| Obsessive-Compulsive Disorder | Separation) | Osteoporosis |
| (OCD) | Recent Trauma | Parkinson's Disease |
| Panic Disorder | History | Tendinitis |
| Posttraumatic Stress Disorder | Self-Compassion | History Of Stroke |
| (PTSD) | Theory Of Mind | History Of Heart |
| Schizophrenia | | Attack |
| Seasonal Affective Disorder | | Body Mass Index |
| Social Anxiety Disorder | | |
| Synesthesia | | |

When the health score is calculated, it may meet one or more thresholds associated with one or more health conditions. In some embodiments, the health score can provide an indication even when a threshold is not met. For example, the health score can be combined with other scores or analyzed along with additional health score collected over a predetermined window of time to provide a better indication of a likelihood of developing a health condition, or being at risk of a health condition.

In operation 420, processor 200 and/or processor 300 identifies the health conditions for which the threshold is met.

In operation 425, processor 200 and/or processor 300 generates a personalized message indicating the health conditions and status from which the threshold is met. The personalized message includes an assessment of all health conditions and status that may be screened, assessed, determined, diagnosed or the like, in a user. The assessment can include a list of the health conditions and status, a chart showing the likelihood that the user has one or more health conditions. In some embodiments, the personalized message can provide a degree of a health condition and status, for example, presenting specific traits that indicate a likelihood of a health condition and status being present, developing, or the like. In some embodiments, the health score can provide an indication of a life experience of the user, for example, satisfaction of life, happiness, or the like.

In operation 430, processor 200 and/or processor 300 provides the personalized message to the output devices 210 (FIG. 2).

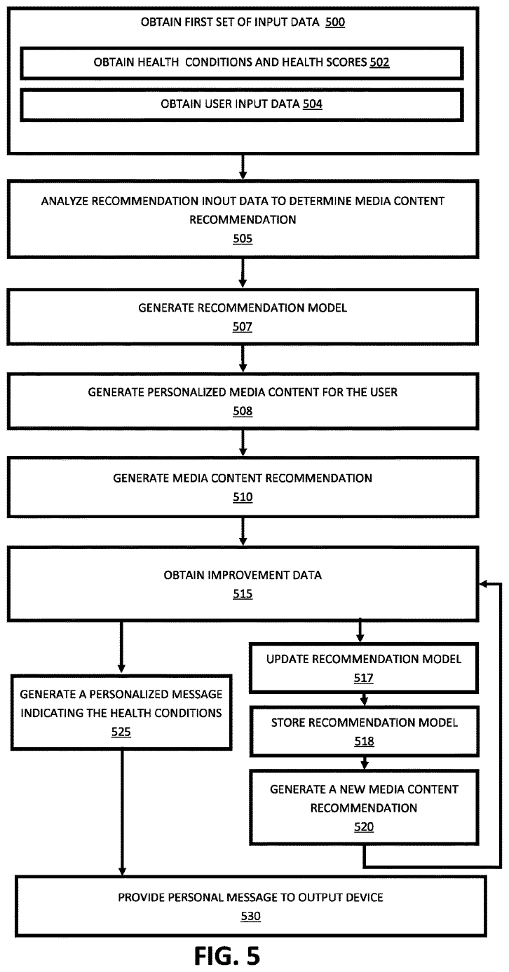

FIG. 5 is a block diagram outlining operations of a method for providing media content for modifying or tracking/monitoring a health condition or status of a user, according to certain exemplary embodiments. In operation 500, processor 200 (FIG. 2) and/or processor 300 (FIG. 3) obtain a first set of input data facilitating generating a prediction of a media content recommendation. In some embodiments, the first set of input data includes the health score and user input data, which can include media content preferences, a current mood of the user, desired health goals of the user, biometric data of the user and the like. In some embodiments, the user provides input data regarding the mood of the user, the needs of the user, the goals of the user, and the like via input devices 205 (FIG. 2).

In operation 505, processor 200 and/or processor 300 analyzes the first set of input data to determine the media content that may improve the health condition and status of the user.

In operation 507, In operation 505, processor 200 and/or processor 300 generate a recommendation model. The consumption of media contact, which may be personalized and recommended to the user, may improve a user's health condition and status. A variety of media attributes, such as calming, inspiring, at a high tempo, or the like, can correspond to different effects on the user. Thus, it is possible to identify media content or generate media content through artificial intelligence, and then recommend it to the user, based on prior known health conditions and status of the user, which may be assessed from their media content preferences or consumption history. For example, music that is cathartic and emotional may tend to improve the health of people who suffer from depression. Whereas music that is overly energetic or happy might have a neutral or even negative affect on the mental health of people who suffer from depression. Furthermore, diversity metrics (e.g., gender or personality) provided by or assigned to the user may inform the recommendations provided by system 100 (FIG. 1). For example, the user who is depressed and extraverted may have a greater improvement in health by listening to music that is cathartic and includes more social elements (e.g., multiple vocalists, choirs), whereas a user that is depressed may have a greater improvement in health by listening to music that is cathartic with little social elements (e.g., only one vocalist or instrumental). A further example: an adult male with depression may have a greater improvement in health by listening to cathartic music with male vocals; while an adult female with depression may have a greater improvement in health by listening to music with female vocals. An even further biological example, music that is calming might decrease cortisol more or less, for those with anxiety disorders compared to those without.

The moment-to-moment health status (e.g., mood state) of a user can be briefly assessed or assigned prior to the media content recommendation, and after the user has engaged with the recommendation, such as after having listened to the song or having watched the video. Therefore, the processor 200 or processor 300 can learn the effects of the recommendations on the individual and improve the accuracy of the model in modifying the health condition and status of the user. The long-term goals, for example, “less lonely” or “lose weight” and/or short term goals of the user, for example, “I would like to feel energized”, “I would like to get in touch with my emotions” or “I would like to focus” of the user can be assessed or assigned prior to the media content recommendation, and after the user has engaged with the media content recommendation, for example, after the user has listened to the song or watched the video.

Furthermore, or in replacement of providing a media content recommendation, the media content can be generated by artificial intelligence and personalized to facilitate modifying the health condition and the status of the user. For example, a user's health condition and status, along with other diversity metrics like gender and personality, and also the current mood and goals of a user, can be provided as inputs to generate a media content output. The generated media content can be recommended to and consumed by the user. The generation of the media content can improve over time based on further learning the effects of the media content on the user.

In optional operation 508, processor 200 and/or processor 300 generate media content that is personalized media content to the user according to first set of input data. In some embodiments, processor 200 and/or processor 300 are configured to generate personalized media content for the user according to attributes and characteristics associated with the media content that can improve the health condition of the user. In some embodiments, processor 200 and/or processor 300 can execute one or more algorithms that utilize either a Long Short-Term Memory (“LSTM”), Recurrent Neural Network (“RNN”), a Generalized Adversarial Network (“GAN”), a simple Markov Chain Model, a Diffusion Music model, a Transformer decoder-based neural network (“TDNet”), or the like. For example, The TDNet facilitates analyzing media content themes that that are of a predetermined time length, such as three seconds, to generate an artificial intelligence new media content. For example, the media content theme is a musical theme that is used to generate a piece of music.

The TDNet can preserve the media content theme and repeat it at a predesignated number of occasions in the generated musical piece. This way the TDNet preserves the media content theme by also remaining creative in generating its spin on the media content theme without deviating from it but rather remaining harmonic to it. By maintaining a cross-attention layer between a Decoder and an Encoder with the use of parallel attention gating the processor 200 and/or processor 300 can maintain the media content theme. Thereby, by determining the media content theme, the processor 200 and/or processor 300 can modify, reinforce, or monitor, the mental health of the user, which can then be monitored and thereby further data can be extracted for the theme from which processor 200, 300 can generate media content that leverage the effect of the media content theme. For example, processor 200 and/or processor 300 are trained on a PIANO909 dataset for Music Arrangement Generation to thereby generate musical themes and music content that can be presented to the user. In some embodiments, the media content themes can be extracted by processor 200 and/or processor 300 executing a DBSCAN clustering algorithm.

In operation 510, processor 200 and/or processor 300 generate a media content recommendation. In some embodiments, the media content recommendation can be a single media content item, such as music content, video content or the like. In other embodiments, the media content recommendation can be a playlist that includes entire songs, videos and/or portions of musical content or video content that are provided continuously to the user to improve the health condition and status of the user.

In operation 515, processor 200 and/or processor 300 records modification data collected by input devices 205. In some embodiments, the user provides modification data via input device 205 that includes the health state, trait, status or the like of the user after consuming the recommended media content. In some embodiments, the modification data provided by the user via input devices 205 can include a rating provided by the user of the effectiveness of the media content recommendation in improving the health condition and status of the user. The user can further provide a written description of the effect of the media content recommendation on the health condition and status of the user.

In some embodiments, input device 205 can include a sensor that measures biometric data of the user, for example through one or more wearable devices 208 (FIG. 2), to monitor the change in the biometrics of the user while the user is engaging the recommended entertainment and afterwards. For example, the sensor can measure a heartrate of the user while consuming to the media content recommendation.

In operation 517, processor 200 and/or processor 300 updates the recommendation model in accordance with the improvement data.

In operation 518, processor 200 and/or processor 300 stores recommendation model, for example, in memory 225 (FIG. 2) and/or memory 310 (FIG. 3).

In operation 520, processor 200 and/or processor 300 generates a new media content recommendation according to the modification data. In some embodiments, the modification data is combined with the first set of input data of step 500 to provide a more accurate recommendation to improve the health condition and status of the user. In some embodiments, the new media content recommendation can include songs and/or playlists for the user to listen to, a video for the user to watch, media generated by artificial intelligence for the user to interact with, a recommendation to play a predetermined musical instrument, a music education or music lessons to engage in, recommend a mental health or physical health professional to interact with, provide a match with a health provider and provide the profile and specialties of the provider to the user, recommend medications and/or supplements, treatment plans, or the like.

In some embodiments, processor 200 and/or processor 300 can repeat steps 515 and 520 to continuously improve and monitor the health condition and status of the user. In some embodiments, processor 200 and/or processor 300 continuously reinforce a learning model according to the modification data and the first set of input data thereby improving a prediction algorithm that provides a more accurate recommendation.

In operation 525, processor 200 and/or processor 300 generates a personalized message indicating the health conditions and status of the user after consumption of recommended media content. In some exemplary embodiments, the personalized message can include a medication prescription and a referral to a medical practitioner for further medical assistance.

In operation 530, processor 200 and/or processor 300 the personalized message to the output devices 210 (FIG. 2).

In the context of some embodiments of the present disclosure, by way of example and without limiting, terms such as ‘operating’ or ‘executing’ also imply capabilities, such as ‘operable’ or ‘executable’, respectively. Conjugated terms such as, by way of example, ‘a thing property’ implies a property of the thing, unless otherwise clearly evident from the context thereof.

The terms ‘processor’ or ‘computer’, or system thereof, are used herein as ordinary context of the art, such as a general purpose processor or a micro-processor, RISC processor, or DSP, possibly comprising additional elements such as memory or communication ports. Optionally or additionally, the terms ‘processor’ or ‘computer’ or derivatives thereof denote an apparatus that is capable of carrying out a provided or an incorporated program and/or is capable of controlling and/or accessing data storage apparatus and/or other apparatus such as input and output ports. The terms ‘processor’ or ‘computer’ also denote a plurality of processors or computers connected, and/or linked and/or otherwise communicating, possibly sharing one or more other resources such as a memory.

The terms ‘software’, ‘program’, ‘software procedure’ or ‘procedure’ or ‘software code’ or ‘code’ or ‘application’ may be used interchangeably according to the context thereof, and denote one or more instructions or directives or circuitry for performing a sequence of operations that generally represent an algorithm and/or other process or method. The program is stored in or on a medium such as RAM, ROM, or disk, or embedded in a circuitry accessible and executable by an apparatus such as a processor or other circuitry. The processor and program may constitute the same apparatus, at least partially, such as an array of electronic gates, such as FPGA or ASIC, designed to perform a programmed sequence of operations, optionally comprising or linked with a processor or other circuitry. The term computerized apparatus or a computerized system or a similar term denotes an apparatus comprising one or more processors operable or operating according to one or more programs.

As used herein, without limiting, a module represents a part of a system, such as a part of a program operating or interacting with one or more other parts on the same unit or on a different unit, or an electronic component or assembly for interacting with one or more other components. As used herein, without limiting, a process represents a collection of operations for achieving a certain objective or an outcome. As used herein, the term ‘server’ denotes a computerized apparatus providing data and/or operational service or services to one or more other apparatuses.

The term ‘configuring’ and/or ‘adapting’ for an objective, or a variation thereof, implies using at least a software and/or electronic circuit and/or auxiliary apparatus designed and/or implemented and/or operable or operative to achieve the objective. A device storing and/or comprising a program and/or data constitutes an article of manufacture. Unless otherwise specified, the program and/or data are stored in or on a non-transitory medium.

In case electrical or electronic equipment is disclosed it is assumed that an appropriate power supply is used for the operation thereof.

The flowchart and block diagrams illustrate architecture, functionality or an operation of possible implementations of systems, methods and computer program products according to various embodiments of the present disclosed subject matter. In this regard, each block in the flowchart or block diagrams may represent a module, segment, or portion of program code, which comprises one or more executable instructions for implementing the specified logical function(s). It should also be noted that, in some alternative implementations, illustrated or described operations may occur in a different order or in combination or as concurrent operations instead of sequential operations to achieve the same or equivalent effect.

The corresponding structures, materials, acts, and equivalents of all means or step plus function elements in the claims below are intended to include any structure, material, or act for performing the function in combination with other claimed elements as specifically claimed. As used herein, the singular forms “a”, “an” and “the” are intended to include the plural forms as well, unless the context clearly indicates otherwise. It will be further understood that the terms “comprises” and/or “comprising” and/or “having” when used in this specification, specify the presence of stated features, integers, steps, operations, elements, and/or components, but do not preclude the presence or addition of one or more other features, integers, steps, operations, elements, components, and/or groups thereof.

As used herein the term “configuring” and/or ‘adapting’ for an objective, or a variation thereof, implies using materials and/or components in a manner designed for and/or implemented and/or operable or operative to achieve the objective.

Unless otherwise specified, the terms ‘about’ and/or ‘close’ with respect to a magnitude or a numerical value implies within an inclusive range of −10% to +10% of the respective magnitude or value. Unless otherwise specified, the terms ‘about’ and/or ‘close’ with respect to a dimension or extent, such as length, implies within an inclusive range of −10% to +10% of the respective dimension or extent. Unless otherwise specified, the terms ‘about’ or ‘close’ imply at or in a region of, or close to a location or a part of an object relative to other parts or regions of the object.

When a range of values is recited, it is merely for convenience or brevity and includes all the possible sub-ranges as well as individual numerical values within and about the boundary of that range. Any numeric value, unless otherwise specified, includes also practical close values enabling an embodiment or a method, and integral values do not exclude fractional values. A sub-range values and practical close values should be considered as specifically disclosed values. As used herein, ellipsis ( . . . ) between two entities or values denotes an inclusive range of entities or values, respectively. For example, A . . . Z implies all the letters from A to Z, inclusively.

The terminology used herein should not be understood as limiting, unless otherwise specified, and is for the purpose of describing particular embodiments only and is not intended to be limiting of the disclosed subject matter. While certain embodiments of the disclosed subject matter have been illustrated and described, it will be clear that the disclosure is not limited to the embodiments described herein. Numerous modifications, changes, variations, substitutions and equivalents are not precluded.

Terms in the claims that follow should be interpreted, without limiting, as characterized or described in the specification.

1. A method for assessing the health of a user, the method performed by at least one processor configured to execute one or more programs stored in a memory, the one or more programs comprising instructions to:
obtain input data associated with the user;
extract at least one characteristic associated with said input data;
determine a health score based on variation of predetermined values;
determine whether said health score satisfies a threshold associated with at least one health condition;
generate a personalized message identifying at least one health condition; and,
provide said personalized message and said health score to an output device.

2. The method according to claim 1, wherein said input data comprises a user musical preference questionnaire input, a playlist history, self-reported measure of musical preferences, user recorded music, demographic information, a user written text about music, entertainment history and preference, media history and preference, music selected for social media posts, music listened to through social media, and a combination thereof.

3. A method according to claim 2, wherein said input mental and physical health assessments and personality questionnaires.

4. The method according to claim 1, wherein said threshold is associated with a mental health condition.

5. The method according to claim 1, wherein said threshold is associated with a neurological condition.

6. The method according to claim 1, wherein said threshold is associated with a social condition.

7. The method according to claim 1, wherein said threshold is associated with a physical condition.

8. The method according to claim 1, wherein said at least one characteristic that describes said input data include values associated with categories selected from the group consisting of genre, artist, popularity, playlist co-occurrence and emotion.

9. The method according to claim 1, wherein said personalized message presents a spectrum of a health condition and presenting where on the spectrum the user is categorized.

10. (canceled)

11. A system configured to assess a health condition of a user, the system comprising:
a user interface having at least one input device and at least one output device;
a client device having at least one processor;
a memory storing one or more programs for execution by said at least one processor, the one or more programs comprising instructions to:
obtain input data associated with the user;
extract at least one characteristic associated with said input data;
determine a health score based on variation of characteristics extracted;
determine whether said health score satisfies a threshold associated with at least one health condition;
generate a personalized message identifying at least one health condition;
and,
provide said personalized message to an output device;

12. The system according to claim 11, wherein said input data comprises a user musical preference questionnaire input, a playlist history, self-reported measure of musical preferences, user recorded music, demographic information, a user written text about music, entertainment history and preference, media history and preference, music selected for social media posts, music listened to through social media, and a combination thereof.

13. (canceled)

14. (canceled)

15. (canceled)

16. (canceled)

17. (canceled)

18. (canceled)

19. The system according to claim 11, wherein said at least one characteristic that describes said input data include values associated with categories selected from the group consisting of genre, artist, popularity, playlist co-occurrence and emotion.

20. The system according to claim 11, wherein said personalized message presents a spectrum of a health condition and presenting where on the spectrum the user is categorized.

21. The system according to claim 20, wherein said health condition is a combination of characteristics associated with one or more health conditions.

22. A system configured to provide a media content recommendation to improve a health of a user, the system comprising:
a user interface having at least one input device and at least one output device;
a client device having at least one processor;
a memory storing one or more programs for execution by said at least one processor, the one or more programs comprising instructions to:
obtain a first set of input data facilitating generating a prediction of media content recommendation;
analyze said first set of input data to determine the media content that may improve the health condition and status of the user;
generate a media content recommendation;
record modification data during and after the user has consumed said media content recommendation;
generate a personalized message indicating the health condition and status of the user after consumption of said media content recommendation; and
providing said personalized message to said at least one output device.

23. The system according to claim 22, wherein said processor is further configured to generate a new media content recommendation according to said modification data.

24. The system according to claim 22, wherein said processor is further configured to personalized media content according to said first set of input data.

25. (canceled)

26. The system according to claim 22, wherein said personalized message provides a medication recommendation to correspond to said media content recommendation.

27. The system according to claim 22, wherein the recommendation can includes a treatment plan for clinical psychology, psychotherapy, and music therapist that is provided directly to a treatment provider.

28. The system according to claim 22, wherein said processor is further configured to:
generate a recommendation model;
update said recommendation model according to said improvement data; and
store said recommendation model.
